(19) United States
(12) Patent Application Publication
(10) Pub. No.: US 2005/0058570 A1
(43) Pub. Date: Mar. 17, 2005

(54) Method and apparatus for surface sterilizing objects

(75) Inventor: Veschi Raffaele, San Paolo (BR)

Correspondence Address: GREENBLUM & BERNSTEIN, P.L.C., 1950 ROLAND CLARKE PLACE, RESTON, VA, 20191, US

(73) Assignee: MONT. EL S.r.L., Sale Marasino (BS) (IT)

(21) Appl. No.: 10/919,321

(22) Filed: Aug. 17, 2004

(30) Foreign Application Priority Data
Aug. 18, 2003 (CH) CH 01410/03

(51) Int. Cl. A61L 2/00

(52) U.S. Cl. 422/22; 422/186

(57) ABSTRACT

Method and apparatus for surface sterilizing objects and for the elimination of bacterial film thereon using an electric and/or magnetic field.

### CROSS-REFERENCE TO RELATED APPLICATIONS

The present application claims priority under 35 U.S.C. §119 of Swiss Patent Application No. 01410/03, filed Aug. 18, 2003, the disclosure of which is expressly incorporated by reference herein in its entirety.

### BACKGROUND OF THE INVENTION

1. Field of the Invention

The present invention concerns methods and apparatus for surface sterilization of objects such as surgical instruments, prosthesis or similar items. In particular, the methods and apparatus according to the present invention permit prevention of the formation as well as provide for the elimination of bacterial films on a wide variety of surfaces, which can be surgical instruments, contact lenses, prosthesis, or similar items.

Using the method according to the present invention, action is aimed at the surfaces concerned by applying an electric field and/or a magnetic field, which are variable over time, and the characteristics of which can be adapted in function of the dimensions of the zone in which the surfaces to be treated are located, in order to eliminate the bacteria and the bio-film possibly present thereon.

In this manner it is possible to treat, and to eliminate, films of bacteria, which have developed characteristics of resistance against other systems, such as e.g. antibiotic or similar systems, in such a manner that an efficient effect of sterilization is obtained also in these cases where the known techniques cannot be applied or would yield poor results.

The present invention also concerns apparatus for implementing the method.

2. Discussion of Background Information

In many situations, sterilization in a simple and economical manner is required for small objects, which can be, e.g., surgical instruments or equipment, prosthesis, or similar items.

For this purpose many methods are known, ranging from the application of solutions containing disinfectant products to treatment with high temperature air or liquids (120-150° C. or more) or using ultraviolet radiation.

Also known are apparatus, which use electromagnetic fields for effecting sterilization of objects, which are described e.g., in U.S. Pat. Nos. 5,632,955, 5,326,530, and 3,753,651 and WO 01/02023, the disclosures of which are incorporated by reference herein in their entireties.

These apparatus, however, concern microwave generators, which aim at sterilizing the whole object, including heating the entire object.

These apparatus prove unsuitable if used for effecting surface sterilization of objects, and also the other known systems known do not always prove suitable for meeting sterilization objectives.

Thus, if objects containing parts of synthetic plastic material, such as, e.g., dental prosthesis, contact lenses or similar items, are to be sterilized, heat cannot be relied on, and in many cases disinfectant solutions cannot be applied, as in some cases these could attack the plastic material forming the object to be sterilized.

In other cases, e.g., in the case of objects presenting complex shapes, systems using ultraviolet radiation can prove unsuitable, and also the application of disinfecting gases does not necessarily yield the desired results.

There is, however, a need to make available mechanism permitting sterilization of surfaces of small objects, effective in completely eliminating the bacteria even in the case of bacteria, which have developed characteristics of specific resistance against certain types of antibiotics.

In the course of its own research, the assignee pursued the hypothesis that application of at least one of electric and magnetic fields could ensure efficient surface sterilization of objects, made of any material desired, with good results and without risk of damaging them.

The experiments carried out thereupon confirmed correctness of this hypothesis and led to the present invention. There present invention provide both an effective apparatus and method which is confirmed by performing a series of experimental tests on bacteria in bio-films performed in laboratories of microbiology.

### SUMMARY OF THE INVENTION

The present application provides a method of sterilizing an object for at least reduction of bacterial film, comprising applying at least one of an electric field and a magnetic field to a surface of the object to at least cause reduction of bacteria. The at least reduction of bacteria can comprise elimination of bacteria.

The applying can comprise applying at least one of an electric field and a magnetic field which is variable over time.

The object can comprise a metallic object which is placed into a metallic container, and a voltage generator can be connected to the metallic object and the metallic container to apply at least one of an electric field and a magnetic field.

The object can be placed in a body representing a cavity for receiving the object; a surrounding cover can be arranged around the body; and a variable current can be circulated in the surrounding cover so that a magnetic field is generated within the cavity.

The object can be inserted into an active zone formed by two opposing, polar expansions of a core made from ferromagnetic material; a surrounding cover can be arranged on the core; and a variable current can be circulated in the surrounding cover so that a magnetic field is generated within the active zone.

The object can comprise a metallic material and/or a plastic material. The object can comprise a slender object, such as tweezers. The object can comprise a surgical instrument or surgical equipment, a prosthesis or a contact lens.

The present invention also provides apparatus for sterilizing an object for reducing or eliminating bacterial film wherein at least one of an electric field and a magnetic field is applied to a surface of the object to cause reduction or elimination of bacteria, said apparatus comprising components as follows: a metallic container presenting a chamber for receiving a metallic object to be treated, a voltage generator; and connecting elements capable of connecting poles of the voltage generator with the metallic object and with the metallic container into which the body is placed.

The present invention also provides an apparatus for sterilizing an object for reducing or eliminating bacterial film wherein at least one of an electric field and a magnetic field is applied to a surface of the object to cause elimination of bacteria, said apparatus comprising components as follows: a body representing a cavity suitable for functioning as an emplacement seat for the insertion of an object to be sterilized; a surrounding cover arranged around the body; and a variable current mechanism capable of circulating a variable current in the surrounding cover so that a magnetic field is generated within the cavity forming the emplacement seat for the object to be sterilized.

The present invention also provides an apparatus for sterilizing an object for reducing or eliminating bacterial film wherein at least one of an electric field and a magnetic field is applied to a surface of the object to cause elimination of bacteria, said apparatus comprising components as follows: a core made from ferromagnetic material presenting two polar expansions arranged opposite each other, the two polar expansions forming an active zone into which the object to be sterilized can be inserted; a surrounding cover arranged on the core; and a variable current mechanism capable of circulating a variable current in the surrounding cover so that a magnetic field is generated within the active zone forming the emplacement room for the object to be sterilized.

### BRIEF DESCRIPTION OF THE DRAWINGS

The present invention is further described in the detailed description that follows by way of non-limiting examples of exemplary embodiments of the present invention, wherein:

FIG. 1 shows a block diagram of a sterilizing unit according to the present invention provided with devices for generating an electric and/or magnetic field;

FIG. 2 shows a schematic view of an apparatus for sterilization according to the present invention;

FIG. 3 shows the electric layout diagram of the apparatus according to the FIG. 2; and

FIG. 4 shows a schematic view of a further preferred version of an apparatus for sterilization according to the principles of the present invention.

### DETAILED DESCRIPTION OF THE PRESENT INVENTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details of the present invention in more detail than is necessary for the fundamental understanding of the present invention, the description taken with the drawings making apparent to those skilled in the art how the several forms of the present invention may be embodied in practice.

The method of sterilization according to the present invention provides that the body, the surface of which is to be sterilized, be subject to an intense electric or magnetic field of suitable intensity and frequency characteristics for a predetermined period of time.

For this purpose, according to the present invention, apparatus for sterilization are provided comprising an emplacement room suitable for taking up the object to be sterilized, and elements suited to create around this object a strong electric and/or magnetic field with determined characteristics of frequency, in such a manner that the bacterial load present on the surface of the object subject to this field is eliminated.

The apparatus can be realized in various configurations depending on the type of objects to be sterilized.

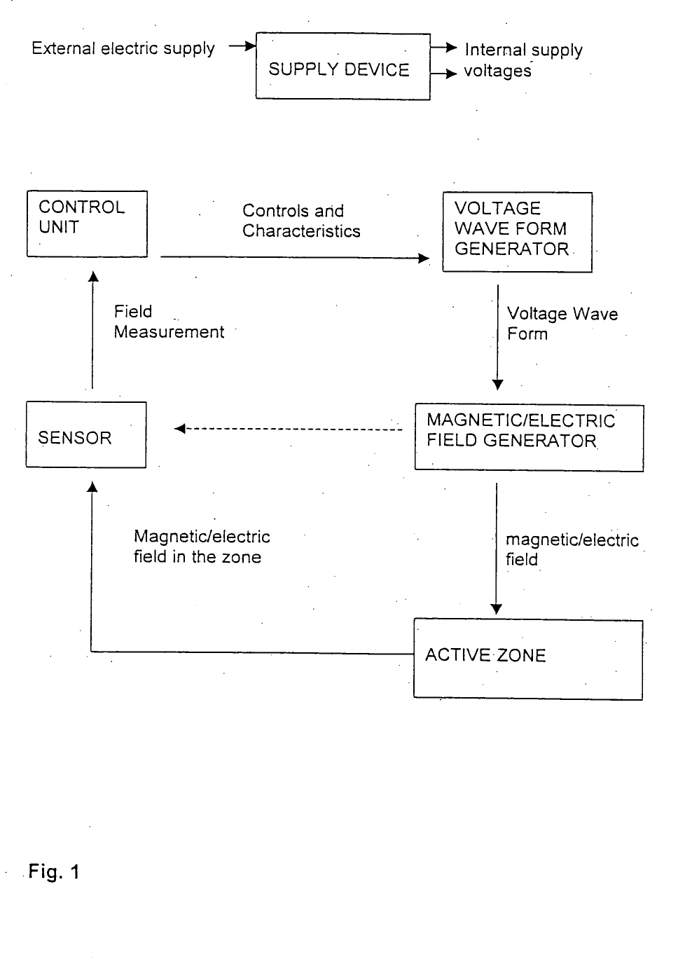

Generally the apparatus for implementing the invention comprises the following functional blocks, such as illustrated in FIG. 1:
1) Supply

The electric/electronic circuit supplies the suitable supply voltage to the other parts of the apparatus from electric energy sources available (household grid, batteries, generator plant, etc.)
2) Wave Form Voltage Generator

This electric/electronic circuit is apt to generate an electric voltage according to rules desired. This voltage is supplied to the magnetic/electric field generator.
3) Control Unit

This electric/electronic unit functions as an interface for the operator, supervising and controlling the other parts of the apparatus. The operator interacts with the control unit for starting and stopping, and for regulating, the magnetic/electric field, and he receives indications concerning the functional state of the apparatus from the unit. This unit controls the wave form generator according to the input given by the operator and in function of the feedback signal from the generator of the magnetic/electric field.
4) Magnetic/Electric Field Generator

This physical system generates a magnetic/electric field, in the active zone or cavity, depending on the voltage supplied by the wave form voltage generator.
5) Feedback Transducer

This sensor is capable of detecting the magnetic/electric field generated in the active zone and to supply a proportional electric feedback signal to the control unit.
6) Active Zone or Cavity

This is the active zone, such as a chamber, room or other zone, in which the surface potentially affected by a bio-film is placed.

The various components of the apparatus, generators, transducers, etc., themselves are known as such to one having ordinary skill in the art, and thus detailed descriptions of the various components are not being further discussed herein. However, one following the guidance provided herein would be capable of including various components in the methods and apparatus of the present invention.

In order to prove the effectiveness of an apparatus of this type, a bio-film was cultivated on a small metallic stick of 2 mm diameter immersed in a nutrient solution in a glass test tube. The test-tube of about 10 mm diameter then was inserted into a metallic cavity. The two poles of a voltage generator were applied to the small metallic stick and the metallic cavity, a radial electric field being generated around the small stick, and acting also onto the bio-film cultivated thereon.

The voltage generator used produced a sinusoidal alternating voltage of peak-to-peak amplitude of 1000 V and at the frequency of 400 Hz. The estimated electric field acting onto the surface of the small stick is of 250 kV/m peak-to-peak.

The results obtained confirm the efficiency of the method, and further tests were carried out with various versions of apparatus according to the present invention.

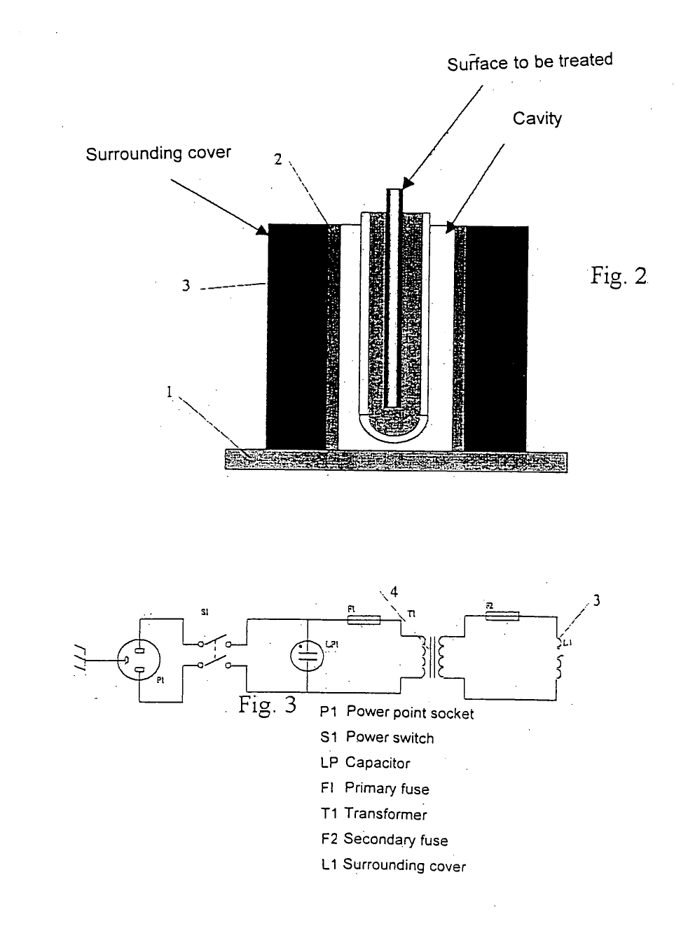

In FIG. 2, an apparatus according to the present invention is shown, capable of generating an axial magnetic field, the circuit diagram of which apparatus is shown in the FIG. 3. This apparatus (FIG. 2) comprises a support 1, to which a plastic tube 2 is soldered, which acts as a support for a surrounding cover 3.

The field generated, extending in the direction of the axis of the tube, is equal to 20 mT peak-to-peak, of sinusoidal form, with a frequency of 50 Hz (corresponding to the grid frequency).

In the cavity inside the tube 2 and the surrounding cover 3, a chamber is created for the placing of objects to be sterilized. They can be objects made from non-ferromagnetic materials, and of one predominant dimension compared to the other ones, such as tweezers, tooth brushes, spatulas, dental mirrors, or similar objects.

The surrounding cover 3 can be supplied, using a transformer 4, directly from the grid current in such a manner that an axial magnetic field alternating at a frequency of 50 Hz (corresponding to the grid frequency) is generated.

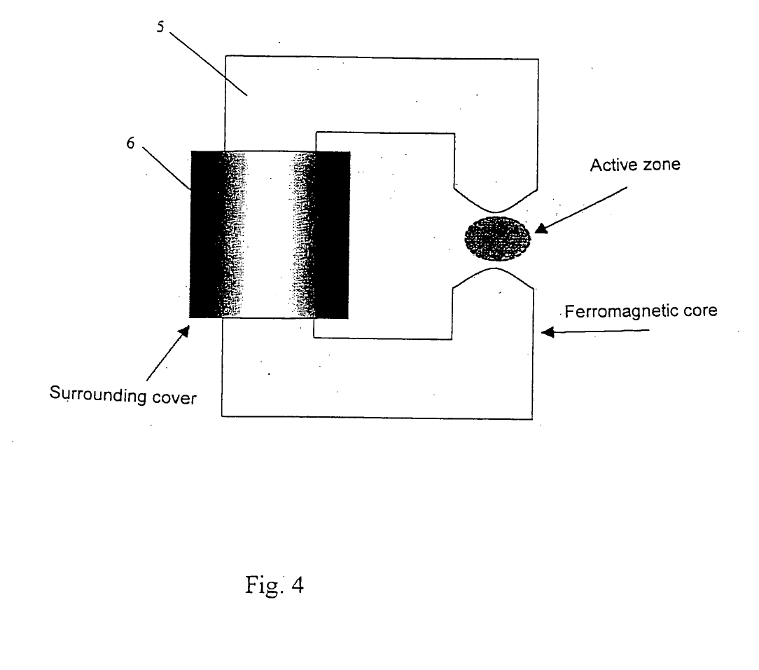

In the FIG. 4, a different form of realization of the apparatus for sterilizing according to the present invention is shown, which can generate a magnetic field in an active zone, such as a chamber or a room, Formed between two polar expansions of a magnetic circuit.

The magnetic circuit is composed of a ferromagnetic “C”-shaped core, designated 5, onto which a surrounding cover 6 is placed.

The empty space within the “C” shape forms the active zone, and the sinusoidal magnetic field, with an amplitude of 250-300 mT peak-to-peak, alternates at a frequency of 50 Hz (corresponding to the grid frequency).

The presence of a ferromagnetic core permits an increase in intensity of the field, even if the active zone cannot be laid out with large dimensions.

This configuration proves particularly favourable for treating small surfaces with very intense fields, e.g., for disinfections of needles or similar items.

The experiments undertaken have confirmed the effectiveness of the system, demonstrating also that, depending on the objects to be treated, the material they are made from, their form and dimensions, it is possible to obtain better results, or within shorter treatment times, if the characteristics of the magnetic or electric fields are suitably adapted.

For this reason, the possibility is provided to make use of treating apparatus with modulated magnetic fields.

Such apparatus, which substantially can be laid out in configurations as illustrated with reference to the preceding examples described, for generating the magnetic field use a surrounding cover on a ferrite core suitable for elevated frequency fields, which is supplied via an electronic circuit oscillating at variable frequency.

With the help of a display, the operator sets the frequency and the amplitude of the oscillation, whereas the electronic arrangement, of a type known as such, takes care of the generation of a corresponding signal, with the possibility of varying it over time.

The method and the apparatus according to the present invention proved effective even if at present the mechanism yielding these results are not yet fully understood. For example and without wishing to be bound by theory, it can be imagined that the energy transmitted to the bacteria during the treatment causes their elimination, presumably by heating the bacteria.

In any case, the experimental tests using bacterial cultures in bio-films carried out by microbiology laboratories have confirmed the effectiveness of the system.

The expert in the field will be able, within the scope of idea of the solution, to provide different forms of realization of the apparatus within the scope of the present invention by following the guidance presented herein.

### EXAMPLES

The invention is explained in more detail in the following on the basis of several exemplary embodiments.

### Example—Microbiologic Test

Bacteria strains used:

Pseudomonas aeruginosa (clinically insulated strain producing bio-film)
Staphylococcus epidermidis (clinically insulated strain producing bio-film)
Staphylococcus aureus ATCC 259271 (strain producing bio-film).
   Substrates and buffer solutions for growing the settled form (bio-film), and for control of, these strains in plankton form:
Brain Hearth Infusion (BHI) (Oxoid)
Buffer: PBS (Sigma products)
NaCl 8 g
Na₂HPO₄ 1.44 g
KH₂PO₄ 0.24 g
KCl 0.2 g

Before each experiment, each bacteria strain was re-controlled using growth on the solid substrate (BHI), and the bacteria species was identified with the help of automated systems (API System).

For each experiment suitably grown bio-film bacteria were used.

Small metallic sticks and polystyrene pellet balls (produced by Bead Corporation USA) were used as support for growing the bio-film thereon. The bacteria strains cited above were inoculated in known quantities (10⁶ cfu) in broth (BHI) in presence of the pre-selected support. The bacteria and the supports remained in the culture broth at 37° C. for at least 5 days. In this manner, the bacteria adhere to the supports, and their colonization develops to form a mature bio-film.

After five days, the supports were subject to the following treatment:

Washing in sterile PBS at least two times (the washing process comprises a vortex treatment for at least one minute) aimed at eliminating free bacterial cells (i.e., of the plankton type);

The support processed in this manner is subject to the pre-selected treatment;

After the treatment, each support was immersed in PBS and exposed to ultrasound for at least two minutes in the Sorvall ultrasound tank.

Owing to this process, the bacterial cells in the surrounding liquid (PBS) are freed from the bio-film; this liquid can be used to quantify the bacteria using counts of the colonies on the solid substrate (see later description). The procedure of ultrasound treatment was repeated three times for each support. After each ultrasound exposure, 100 micro-liters of PBS containing the bacterial cells loosened from the bio-film were placed on Petri dishes containing solid BHI in order to evaluate the number of bacteria that had survived the treatment. The number of surviving bacteria was determined by counting the colonies on the solid substrate after 24 hours of incubation at 37° C. Furthermore, for better control, every support exposed three times to the ultrasound treatment is placed in the culture broth liquid (BHI) for 24 hours at 37° C. in order to check for possible presence of bio-film still alive but not taken off by the ultrasound treatment.

This treatment was repeated every day for 5 days (for 6 days for the last experiment).

### Results of the Experiments

Experiment Using Electric Fields

| | Day 1 | | Day 2 | | Day 3 | | Day 4 | | Day 5 | | Day 6 | | Day 7 | |
|---|---|---|---|---|---|---|---|---|---|---|---|---|---|---|
| Minutcs/ | 40′ | 2′ | 40′ | 2′ | 40′ | 2′ | 40′ | 2′ | 40′ | 2′ | 40′ | 2′ | 40′ | 2′ |
| Volts | 1000 | 12000 | 1000 | 12000 | 1000 | 12000 | 1000 | 12000 | 1000 | 12000 | 1000 | 12000 | 1000 | 12000 |
| Staphyloc. | +++ | +++ | 50% | 50% | − | − | − | − | − | − | − | − | − | − |
| epidermidis | | | | | | | | | | | | | | |
| Staphyloc. | +++ | +++ | 50% | 50% | − | − | − | − | − | − | − | − | − | − |
| aureus | | | 80% | | | | | | | | | | | |
| Pseudom. | +++ | +++ | +++ | +++ | +++ | ? | +++ | +++ | ++− | ++− | ++− | ++− | ++− | ++− |
| aeruginosa | | | | | | | | | | | | | | |

Experiment Using the First Magnetic Apparatus (Small Stick Immersed at the Center) Magnetic Field, Gram Positive and Gram Negative

| | Day | Day | Day | Day | Day | Day | |
|---|---|---|---|---|---|---|---|
| | 1 | 2 | 3 | 4 | 5 | 6 | Control |
| Pseudomon. | +++ | ++ | ++− | +− − | ++− | ++− | +++ |
| aeruginosa | | | | | | | |
| Staphylo. | ++− | ++− | −+ | −+ | +− | +− | +++ |
| aureus | | | | | | | |
| Staphylococcus. | +− − | − − − | − − − | − − − | − − − | − − − | +++ |
| epidermidis | | | | | | | |

Experiment Using Second Magnetic Apparatus (Pellets Immersed)

| | | | | | | | | Control |
|---|---|---|---|---|---|---|---|---|
| | | | | | | | Con- | pellets |
| | Day | Day | Day | Day | Day | Day | trol | PBS be- |
| | 1 | 2 | 3 | 4 | 5 | 6 | strain | fore test |
| Pseudomon. | +++ | +++ | +++ | +++ | +++ | +++ | +++ | +++ |
| aeruginosa | | | | | | | | |
| Staph. | +++ | +++ | +++ | +++ | +++ | +++ | +++ | +++ |
| aureus | | | | | | | | |
| Staph. | − | − | − | − | − | − | ++ | ++ |
| Epidermidis | | | | | | | | |

Legend:
+++ 1200 colonies per Petri dish
++ 600 colonies per Petri dish
+ 300 colonies per Petri dish
+− 150 colonies per Petri dish
−+ 100 colonies per Petri dish
− Absence of growth also in the control broth of the support after ultrasound treatment

It is noted that the foregoing examples have been provided merely for the purpose of explanation and are in no way to be construed as limiting of the present invention. While the present invention has been described with reference to an exemplary embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

1. A method of sterilizing an object for at least reduction of bacterial film, comprising applying at least one of an electric field and a magnetic field to a surface of the object to at least cause reduction of bacteria.

2. The method according to claim 1, wherein the at least reduction of bacteria comprises elimination of bacteria.

3. The method according to claim 1, wherein the applying comprises applying at least one of an electric field and a magnetic field which is variable over time.

4. The method according to claim 3, wherein the at least reduction of bacteria comprises elimination of bacteria.

5. The method according to claim 1, wherein the object comprises a metallic object which is placed into a metallic container, and a voltage generator is connected to the metallic object and the metallic container to apply at least one of an electric field and a magnetic field.

6. The method according to claim 1, wherein the object is placed in a body representing a cavity for receiving the object; a surrounding cover is arranged around the body; and a variable current is circulated in the surrounding cover so that a magnetic field is generated within the cavity.

7. The method according to claim 1, wherein the object is inserted into an active zone formed by two opposing, polar expansions of a core made from ferromagnetic material; a surrounding cover is arranged on the core; and a variable current is circulated in the surrounding cover so that a magnetic field is generated within said active zone.

8. The method according to claim 1, wherein the object comprises a metallic material.

9. The method according to claim 1, wherein the object comprises a plastic material.

10. The method according to claim 1, wherein the object comprises a slender object.

11. The method according to claim 10, wherein the object comprises tweezers.

12. The method according to claim 1, wherein the object comprise a surgical instrument or surgical equipment.

13. The method according to claim 1, wherein the object comprises a prosthesis.

14. The method according to claim 1, wherein the object comprises a contact lens.

15. The method according to claim 2, wherein the object comprises a metallic material.

16. The method according to claim 2, wherein the object comprises a plastic material.

17. The method according to claim 2, wherein the object comprises a slender object.

18. An apparatus for sterilizing an object for reducing or eliminating bacterial film wherein at least one of an electric field and a magnetic field is applied to a surface of the object to cause reduction or elimination of bacteria, said apparatus comprising components as follows:
a metallic container presenting a chamber for receiving a metallic object to be treated,
a voltage generator; and
connecting elements capable of connecting poles of said voltage generator with the metallic object and with the metallic container into which said body is placed.

19. An apparatus for sterilizing an object for reducing or eliminating bacterial film wherein at least one of an electric field and a magnetic field is applied to a surface of the object to cause elimination of bacteria, said apparatus comprising components as follows:
a body representing a cavity suitable for functioning as an emplacement seat for the insertion of an object to be sterilized;
a surrounding cover arranged around said body; and
a variable current mechanism capable of circulating a variable current in said surrounding cover so that a magnetic field is generated within said cavity forming the emplacement seat for the object to be sterilized.

20. An apparatus for sterilizing an object for reducing or eliminating bacterial film wherein at least one of an electric field and a magnetic field is applied to a surface of the object to cause elimination of bacteria, said apparatus comprising components as follows:
a core made from ferromagnetic material presenting two polar expansions arranged opposite each other, said two polar expansions forming an active zone into which the object to be sterilized can be inserted;
a surrounding cover arranged on said core; and
a variable current mechanism capable of circulating a variable current in said surrounding cover so that a magnetic field is generated within said active zone forming the emplacement room for the object to be sterilized.
